(19) United States
(12) Patent Application Publication
(10) Pub. No.: US 2007/0088419 A1
(43) Pub. Date: Apr. 19, 2007

(54) Conductive pad assembly for electrical therapy device

(76) Inventors: Mark A. Fiorina, Elm Grove, WI (US); Mark Fiorina, Elm Grove, WI (US)

Correspondence Address: Philip G. Meyers;Philip G. Meyers Law Office, Suite 300, 1009 Long Prairie Road, Flower Mound, TX, 75022, US

(21) Appl. No.: 11/249,181

(22) Filed: Oct. 13, 2005

(51) Int. Cl. A61N 1/00

(52) U.S. Cl. 607/152

(57) ABSTRACT

A pad assembly according to the invention is used for mounting a current generator for applying a therapeutic electrical current to an area of human skin. Such an assembly includes a pad comprising a thin, flexible, stretchable plastic film that conforms to human skin, which pad has electrical paths printed on an underside thereof with electrically conductive ink. Each of the paths include a terminal and a conductive contact for connection to a current generator. A pair of layers of an electrically conductive adhesive such as a hydrogel are disposed over the terminals at spaced positions for conducting electrical current to human skin underlying the terminals. A fixture secured to the pad is configured for mounting the current generator a manner effective to conduct current from the current generator to the terminals of the pad.

### TECHNICAL FIELD

The invention relates to devices for applying small amounts of electrical current to the skin for therapeutic purposes.

### BACKGROUND OF THE INVENTION

Devices for treating the human body by application of electrical current to the skin are in widespread use. These include transcutaneous electrical nerve stimulation (TENS) devices as well as more recently developed microcurrent therapy devices such as discussed in Powell U.S. Pat. Nos. 6,408,211 and 6,606,519. As the '519 patent exemplifies, a common configuration for such devices includes a conductive pad having a small microcurrent therapy device powered by a battery mounted thereon. The conductive pad may be mounted on the underside of a conventional bandage or flexible carrier and connected to the microcurrent therapy device by wires or conductive paths; see, e.g., Michelson et al. U.S. Pat. No. 6,445,955. The electrodes are covered with an electrically conductive hydrogel, and the bandage or carrier is secured to the skin by an adhesive. It is common for the electric therapy device to be mounted at the center of the carrier so that its conductive terminals are in contact with the leads that carry the current to the electrodes located on wing portions of the carrier.

Recent attention has focused on the current delivery device with relatively little attention to the conductive bandage or carrier pad for attaching the device to the skin. One pad assembly presently marketed by Cyclotec comprises a fabric 4-wing bandage having silver ink applied to the underside thereof, and hydrogel adhesive over the silver ink. The fabric bandage material bunches easily, becomes dirty quickly and must generally be thrown away after one or a few uses. The current generator unit is mounted on a pair of plastic sheets disposed over a central hole in the bandage. The top sheet is a disk of about the same diameter as the hole. End portions of the silver ink paths extend onto the top disk, which has a set of apertures therein by which the current generator can contact the paths. The bottom sheet comprises a central disk with arm portions covering the silver ink paths up to the start of the hydrogel layers. The plastic sheets thereby enclose the inner end portions of the paths on both sides. This type of fixture relies on adhesives and lacks durability.

An improved pad assembly for use with a therapeutic current generator needs to be suitable for extended wearing, conform in the manner of a flexible bandage, yet also be repositionable/reusable. The present invention addresses these needs.

### SUMMARY OF THE INVENTION

A pad assembly according to the invention is used for mounting a current generator for applying a therapeutic electrical current to an area of human skin. Such an assembly includes a pad comprising a thin, flexible, stretchable plastic film that conforms to human skin, which pad has electrical paths printed on an underside thereof with electrically conductive ink. Each of the paths include a terminal and a conductive contact for connection to a current generator. A pair of layers of an electrically conductive adhesive such as a hydrogel are disposed over the terminals at spaced positions for conducting electrical current to human skin underlying the terminals. A fixture secured to the pad is configured for mounting the current generator a manner effective to conduct current from the current generator to the terminals of the pad.

According to a second aspect of the invention, a pad assembly for mounting a current generator for applying a therapeutic electrical current to an area of human skin includes:

a pad comprising a thin, flexible plastic film having a pair of wings extending from a midportion having a central opening therethrough;

a pair of first electrical paths disposed on an underside of the pad, each of which paths include an inner contact proximate the central opening for connection to a current generator, a terminal on one of the wings, and a connecting portion extending from the inner contact to the terminal;

a pair of layers of electrically conductive adhesive covering the terminals for conducting electrical current to an area of human skin underlying the terminals, and for replaceably securing the pad assembly to human skin; and

a fixture mounted on the pad over the central opening, the fixture including portions disposed on opposite sides of the central opening, means for securing a current generator to the fixture, and means for conducting current from the current generator to the inner contacts of the first paths.

The means for conducting current from the current generator to the inner contacts of the first paths may comprise a reversing sheet of plastic film having a second pair of electrical paths formed on one side thereof. Each second path is configured for face to face contact with the inner contact of one of the first paths and with a contact of the current generator extending into the central opening of the pad. In a preferred form of the invention, the fixture comprises a base having a set of pins extending from an inner face thereof and a mount having openings on an inner face thereof for receiving the pins of the base. The pins extend through aligned holes in the pad and in the reversing sheet of plastic film having the second pair of electrical paths thereon, whereby the fixture clamps the reversing sheet to the pad from opposite sides of the pad.

The invention further provides a method for making a pad assembly according to the foregoing embodiments. This and other aspects of the invention are further discussed in the detailed description that follows.

### BRIEF DESCRIPTION OF THE DRAWINGS

FIG. 1 is an exploded perspective view of a conductive pad assembly according to the invention;

FIG. 2 is an exploded top view of the pad and reversing disk shown in FIG. 1;

FIG. 3 is a top view of the pad and reversing disk of FIG. 2 with the disk mounted beneath the pad;

FIG. 4 is a cross section taken along the line 4-4 in FIG. 3;

FIG. 5 is a cross section of a pad according to the invention having additional ink layers printed thereon; and

FIG. 6 is a top view of a pad assembly according to the invention having four wings.

### DETAILED DESCRIPTION

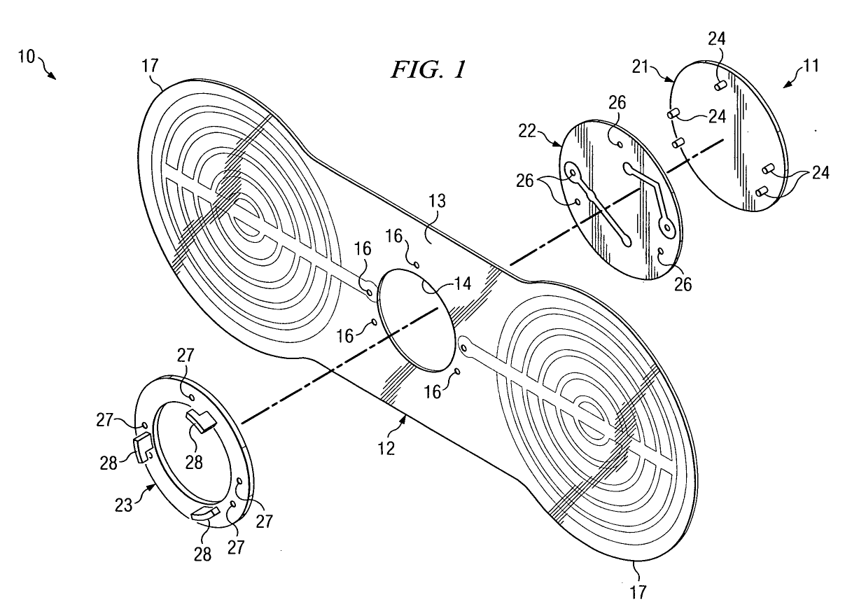

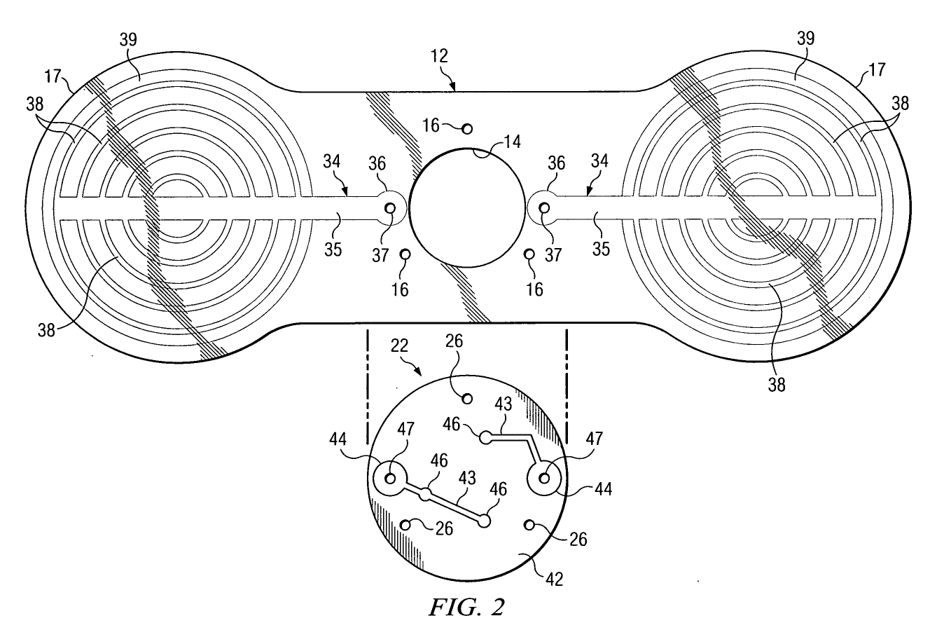

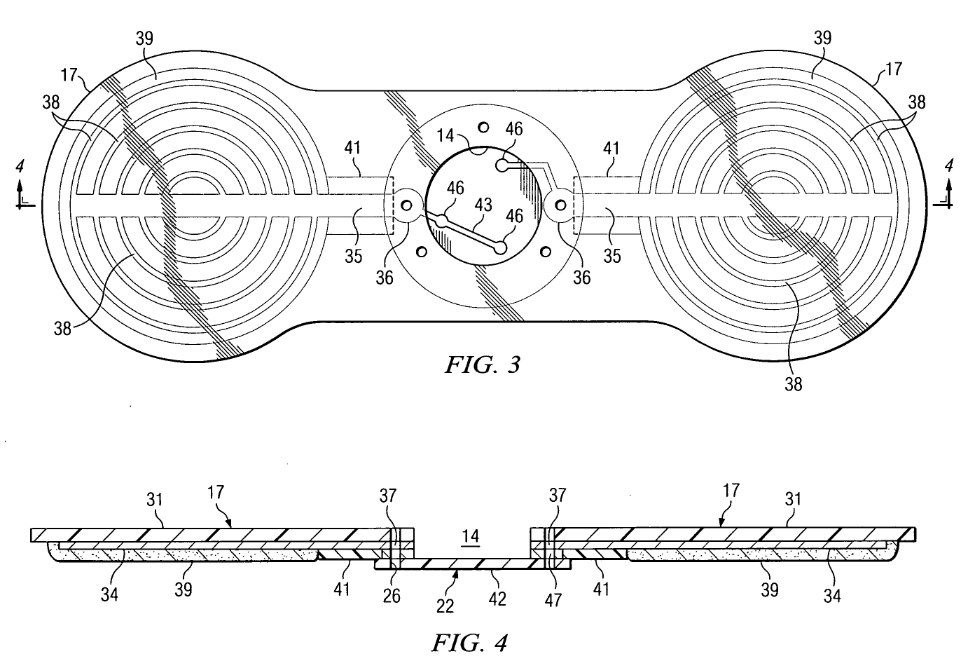

Referring now to FIGS. 1-4, a pad assembly 10 according to the invention includes a mounting fixture 11 that is removably mounted on a flexible carrier or pad 12. Device 10 is configured for attachment to a battery-powered current generator made by Cyclotec having a disk-shaped housing that mounts on fixture 11. The current generator may, for example, be a Cyclotec CT1 unit powered by a 3 v lithium battery. For such devices, see Michelson et al. U.S. Pat. No. 6,445,955 and Publication No. 20020193844, the contents of which are incorporated by reference herein. Pad 12 has a midportion 13 provided with a round central opening 14 and a pentagonal formation of pin hole openings 16 ranged about central opening 14. A pair of identical wings 17 extend from midportion 13 in opposite directions to provide positive and negative terminals for the current generator.

Fixture 11 is mounted over central opening 14. Fixture 11 includes a disk-shaped plastic base 21, a disk-shaped reversing sheet 22, and a mount in the form of a plastic ring 23. A set of short plastic pins 24 molded on the inner face of base 21 extend through aligned holes 26 in reversing disk 22 and pin hole openings 16 into holes 27 on the underside of ring 23. Pins 24 are secured in holes 27 by heat staking or sonic bonding. Three L-shaped projections 28 on the outside of ring 23 engage corresponding recesses in the housing of the current generator so that the current generator can be pressed over projections 28 and the twisted to lock the current generator in place in a manner well known in the art. In this manner, fixture 20 secures the current generator to the outside of pad 12 in a manner that provides electrical conductivity between the terminals of the current generator and the positive and negative electrodes of pad 12.

Pad 12 has a multilayer construction and is preferably manufactured as follows. While many plastic, fabric and paper materials are available for use in making bandages to be applied to the skin, the properties of such materials must be determined to ensure suitability for use in the present invention. A base layer 31 of pad 12 is a plastic film that is flexible, stretchable, and printable, and conforms to human skin. Optical transparency is highly preferred because it can make the pad assembly 10 less conspicuous when in use, or permit custom printing or decoration. For this purpose, perfect transparency is not required, but the film should be sufficiently transparent that underprinting on the film as described hereafter is readable. Films made of vinyl, rubbers, nylon, polyester, polypropylene and polyethylene can be used. However, many commercially available rubbers such as Santoprene are not ideal because they are opaque and cannot be printed on without a pretreatment. Polypropylene films tend to be stiff and have poor compliance to the skin. Vinyl films are thus preferred for use in the invention. The thickness of the film influences the pad's stretchability, flexibility and ability to conform to the skin without stiffness or resistance. Film thicknesses in the range from about 0.001 to 0.125 inch are thus preferred when using films of the foregoing plastics.

In this example, layer 31 is a transparent vinyl film. A silver-based conductive ink is used to print electrical paths 34 onto the underside of film 31. One or more ink layers containing printing, decorations or a background may be interposed between paths 34 and film 31 as described further below. Paths 34 are preferably symmetrical to one another and include inner contacts 36 having holes 37 therethrough and configured for alignment with associated pins 24 on opposite sides of central opening 14. The distal portion of each path 34 forming the terminals includes a series of concentric annular bands 38 that distribute current over an area of the skin underlying each wing 17. Bands 38 are covered by circular layers 39 of electrically conductive hydrogel. A connecting portion 35 of each path 34 is covered with a rectangular patch 41 of dielectric film so that only contact 36 is uncovered. Patch 41 may be laminated onto or adhesively bonded over the connecting portion 35 of path 34 and may be made of the same plastic as base layer 31, or may be a layer of electrically insulating ink printed over the connecting portions 35.

A variety of conductive hydrogels for attachment of electrodes to human are commercially available. All of these hydrogels have adhesive qualities and can be used to adhere pad assembly 10 according to the invention to the skin. However, different hydrogel formulations vary in stickiness and in their ability to resist moisture and oils found in human skin. A preferred hydrogel for use in the invention is KM30B made by Katecho, Inc., which resists deterioration and can permit repeated removal and replacement of the pad on the skin. If additional adhesion is needed, the hydrogel layer 39 can be extended to cover portions of the middle of pad 12, or a layer of another adhesive, such as used in conventional bandages, can be applied on the underside of pad 12 around the periphery of layer 39 and on midportion 13.

Reversing disk 22 is positioned to underlie central opening 14. Disk 22 includes a base layer 42 made of the same thin plastic film as base layer 31, and a pair of spaced, electrically conductive paths 43, which may be printed from the same silver ink as paths 34. Paths 43 have enlarged outer conductive contacts 44 and inner enlarged conductive contacts 46. Contacts 44 face upwardly and have central holes 47 therethrough in alignment with the associated holes 26.

Inner conductive contacts 46 are exposed through central opening 14 and are positioned to contact corresponding positive and negative terminals of the current generator once the current generator has been installed on fixture 11 as described above. Fixture 11 clamps disk 22 and pad 12 firmly in a manner that maintains electrical connections without need for soldering.

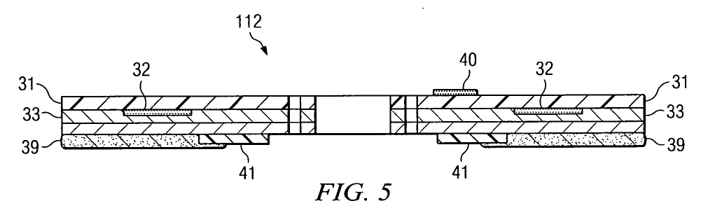

FIG. 5 illustrates an alternative embodiment of a pad 112 according to the invention wherein film 31 is underprinted with black lettering (as by silk screening) 32, for example, the manufacturer's name printed so that it will be readable when viewed through film 31, or a machine readable code such as a bar code identifying the device and/or the wearer. A background layer 33, in this example a white ink, gives the entire pad a white color except at the printed areas. Overprinting such as at 40 is also possible.

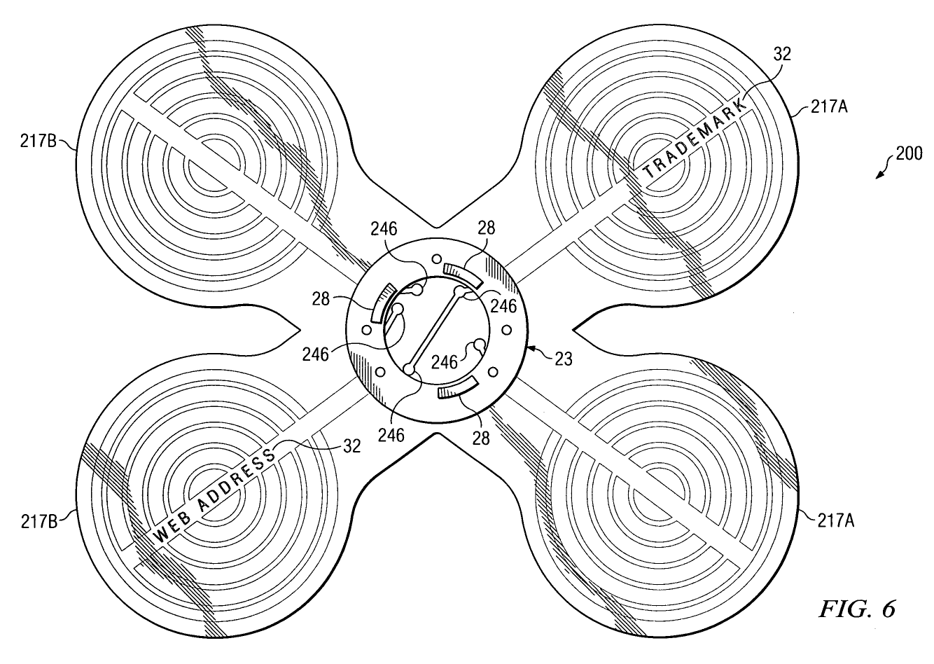

FIG. 6 illustrates a four electrode version of a device 200 according to the invention. The construction is substantially the same as described for FIGS. 1-5, except that the number of inner contacts 246 and the corresponding connections is at least four, one for each wing 217. The current generator may, for example, be configured to create positive electrodes at wings 217A and negative electrodes at each of wings 217B.

A pad assembly according to the invention can be readily made by molding the base layer of the pad or cutting it to the desired shape from a sheet, and then progressively building up successive layers by lamination or screen printing, as appropriate. The reversing disk is similarly printed with conductive pathways and cut to size. The parts of the mounting fixture may be injection molded. Assembly is carried out by positioning the pad and reversing disk onto the pins of the fixture base, ensuring proper alignment, followed by heat staking the mounting ring to the exposed pin ends. The current generator may then the mounted by placing it over the L-shaped projections on the mounting ring and twisting so that the circumferential arms of the L-shaped projections become mechanically interlocked with grooves on the underside of the housing of the current generator unit.

While certain embodiments of the invention have been illustrated for the purposes of this disclosure, numerous changes in the method and apparatus of the invention presented herein may be made by those skilled in the art. For example, any suitable mechanical fastening system, such as screws, may be used to secured the current generator to the outside of mount 23, or mount 23 may be integral with the housing of the current generator. Similarly, it is not essential for the mounting fixture to be attached at the center of the pad. The reversing disk is a convenient and inexpensive way to conduct current from the downwardly facing current generator to the downwardly facing inner contacts of the paths, but wires or printed circuits formed on the base of the fixture could be used for this purpose as well. These and other variations are within the scope of the invention as defined in the appended claims. It should be further understood that the current generator described herein is known in the art and is referenced herein as part of the environment with which the pad assembly of the invention interacts.

1. A pad assembly for mounting a current generator for applying a therapeutic electrical current to an area of human skin, comprising:
a pad comprising a thin, flexible plastic film having electrical paths printed on an underside thereof with electrically conductive ink, each of which paths include a terminal and a contact for connection to a current generator, the pad further having a pair of layers of an electrically conductive adhesive disposed over the terminals at spaced positions for conducting electrical current to human skin underlying the terminals; and
a fixture secured to the pad configured for mounting a current generator on the fixture a manner effective to conduct current from the current generator to the terminals of the pad.

2. The pad assembly of claim 1, wherein the thin, flexible plastic film is made of a material selected from the group consisting of vinyl, rubber, nylon, polyester, polypropylene and polyethylene.

3. The pad assembly of claim 2, wherein the thin, flexible plastic film is made of vinyl.

4. The pad assembly of claim 3, wherein the thin, flexible plastic film has a thickness in the range of about 0.001 to 0.125 inch.

5. The pad assembly of claim 1, wherein the thin, flexible plastic film has a pair of wings extending from a midportion having a central opening therethrough, and the fixture is configured for mounting over the central opening.

6. The pad assembly of claim 1, wherein the electrically conductive adhesive is a hydrogel.

7. The pad assembly of claim 1, wherein the thin, flexible plastic film is transparent and has a layer of underprinting thereon readable through the transparent film.

8. A pad assembly for mounting a current generator for applying a therapeutic electrical current to an area of human skin, comprising:
a pad comprising a thin, flexible, stretchable plastic film that conforms to human skin having a pair of wings extending from a midportion having a central opening therethrough;
a pair of first electrical paths disposed on an underside of the pad, each of which paths include a inner contact proximate the central opening for connection to a current generator, a terminal on one of the wings, and a connecting portion extending from the inner contact to the terminal;
a pair of layers of electrically conductive adhesive covering the terminals for conducting electrical current to an area of human skin underlying the terminals, and for replaceably securing the pad assembly to human skin; and
a fixture mounted on the pad over the central opening, the fixture including portions disposed on opposite sides of the central opening, means for securing a current generator to the fixture, and means for conducting current from the current generator to the inner contacts of the first paths.

9. The pad assembly of claim 8, wherein the thin, flexible plastic film is made of a material selected from the group consisting of vinyl, rubber, nylon, polyester, polypropylene and polyethylene, and has a thickness in the range of about 0.001 to 0.125 inch, the first electrical paths are printed on an underside of the thin, flexible plastic film with an electrically conductive ink, and the electrically conductive adhesive is a hydrogel.

10. The pad assembly of claim 8, further comprising a layer of dielectric material covering the connecting portion of each path.

11. The pad assembly of claim 8, wherein the means for conducting current from the current generator to the inner contacts of the first paths comprises a reversing sheet of plastic film having a second pair of electrical paths formed on one side thereof, wherein each second path is configured for face to face contact with the inner contact of one of the first paths and with a contact of the current generator extending into the central opening of the pad.

12. The pad assembly of claim 1 1, wherein the fixture further comprises a base having a set of pins extending from an inner face thereof and a mount having openings on an inner face thereof for receiving the pins of the base, whereby the pins extend through aligned holes in the pad and in the sheet of plastic film having the second pair of electrical paths thereon, whereby the fixture clamps the reversing sheet to the pad from opposite sides of the pad.

13. The pad assembly of claim 12, wherein the mount has a series of L-shaped projections on an outer face thereof configured for mechanical engagement with the current generator for removably securing the current generator to the mount.
